(19) United States
(12) Patent Application Publication
(10) Pub. No.: US 2002/0123470 A1
(43) Pub. Date: Sep. 5, 2002

(54) Use of aminoglycosides to treat genetic ophthalmic diseases that are associated with premature termination mutations

(75) Inventor: Abbot F. Clark, Arlington, TX (US)

Correspondence Address: Alcon, Inc., c/o Alcon Research, Ltd., Patrick M. Ryan(Q-148), R&D Councel, 6201 So. Freeway, Fort Worth, TX, 76134-2099, US

(73) Assignee: Alcon, Inc.

(21) Appl. No.: 10/085,322

(22) Filed: Feb. 28, 2002

Related U.S. Application Data
(60) Provisional application No. 60/273,691, filed on Mar. 5, 2001.

(51) Int. Cl. A61K 31/704; A61K 31/7008

(52) U.S. Cl. 514/36; 514/41; 514/37

(57) ABSTRACT

Aminoglycoside antibiotics are used to treat genetic ophthalmic diseases caused by premature “stop” mutations.

This application claims priority from co-pending U.S. Provisional Application, U.S. Serial No. 60/273,691, filed Mar. 5, 2001.

### BACKGROUND OF THE INVENTION

The present invention relates generally to methods for treating certain genetic ophthalmic diseases. In particular, the invention relates to the use of aminoglycosides to treat ophthalmic diseases, including glaucoma, caused by premature termination (“stop”) mutations in the disease gene, such as in the glaucoma genes MYOC (GLC1A), CYP1B1 (GLCBA), and FOXC1 (FKHL7).

Glaucoma is a group of ocular disorders, characterized by degeneration of the optic nerve. It is one of the leading causes of blindness worldwide. One major risk factor for developing glaucoma is family history. A number of different inherited forms of glaucoma have been described.

Primary congenital or infantile glaucoma is an inherited disorder that is characterized by an improper development of the aqueous outflow system of the eye, which leads to elevated intraocular pressure, enlargement of the globe or cornea (i.e., buphthalmos), damage to the optic nerve, and eventual visual impairment.

Primary open angle glaucoma (POAG) is a common disorder characterized by atrophy of the optic nerve resulting in visual field loss and eventual blindness. POAG has been divided into two major groups, based on age of onset and differences in clinical presentation.

Juvenile-onset POAG usually manifests in late childhood or early adulthood. Its progression is rapid and severe, with high intraocular pressure. This type of POAG is poorly responsive to medical treatment, and usually requires ocular surgery.

Adult- or late-onset POAG is the most common type of glaucoma. It is milder and develops more gradually than juvenile-onset POAG, with variable onset usually after the age of 40. This type of POAG is associated with slight to moderate elevation of intraocular pressure, and often responds satisfactorily to regularly monitored medical treatment. Unfortunately, this disease may not be detected until after irreversible damage to the optic nerve has already occurred because it progresses gradually and painlessly.

U.S. Pat. No. 5,830,661 discloses methods for the diagnosis and treatment of glaucoma associated with mutations in the CYP1B1 gene.

U.S. Pat. No. 5,925,748 discloses diagnostic methods for glaucoma associated with mutations in the GLC1A gene.

“Stop” mutations in the glaucoma genes GLC1A and in the CYP1B1 are known to exist. Stone, et al., “Identification of a Gene That Causes Primary Open Angle Glaucoma,” Science Vol. 275(5300) pp. 668-670 (1997); Adam et al., “Recurrent Mutations in a Single Exon Encoding the evolutionarily Conserved Olfactomedin-homology Domain of TIGR in Familial Open-Angle Glaucoma,” Hum Mol Genet, Vol. 6(12) pp. 2091-7 (1997); Alward et al., “Clinical Features Associated with Mutations in the Chromosome 1 Open-Angle Glaucoma Gene (GLC1A); New England J. Med., Vol. 338(15); pp. 1022-7 (1998); Mardin et al., “A GLC1A gene Gln368Stop Mutation in a Patient with Normal-Tension Open-Angle Glaucoma,” J. Glaucoma, Vol. 8(2); pp. 154-6 (1999); Angius et al., “Myocilin Gln368stop Mutation and Advanced Age as Risk Factors for Late-Onset Primary Open-Angle Glaucoma,” Arch Ophthalmology, Vol. 118(5); pp. 674-9 (2000); Shimizu et al., “Age-dependent Prevalence of Mutations at the GLC1A Locus in Primary Open-Angle Glaucoma,” Am J. Ophthalmol, Vol. 130(2); pp. 165-77 (2000); Kakiuchi et al., “A Novel Truncating Mutation of Cytochrome P4501B1 (CYP1B1) Gene in Primary Infantile Glaucoma,” Am J. Ophthalmol., Vol. 128(3); pp. 370-2 (1999). The most prevalent GLC1A mutation identified to date is a stop mutation at codon 368, which is responsible for approximately 1.6-2% of POAG. Fingert et al., “Analysis of Myocilin Mutations in 1703 Glaucoma Patients from Five Different Populations,” Hum Mol Genet, Vol. 8(5); pp. 899-905 (1999)

The use of aminoglycosides to treat muscular dystrophy and cystic fibrosis caused by stop mutations in certain genes has been suggested. Bedwell, et al., “Suppression of a CFTR premature stop mutation in a bronchial epithelial cell line,” Nat Med. 3(11):1280-1284 (1997) and Howard, et al., “Aminoglycoside antibiotics restore CFTR function by overcoming premature stop mutations,” Nat Med. 2(4):467-469 (1996), disclose the use of aminoglycosides to treat cystic fibrosis caused by mutations in the cystic fibrosis transmembrane conductance regulator protein. Barton-Davis, et al., “Aminoglycoside antibiotics restore dystrophin function to skeletal muscles of mdx mice,” J. Clin. Invest. 104(4):375-381 (1999), discloses the use of aminoglycosides to overcome stop mutations in Duchenne muscular dystrophy. The in vitro and in vivo results reportedly obtained with the aminoglycoside gentamicin raise “the possibility of a novel treatment regimen for muscular dystrophy and other diseases caused by premature stop codon mutations.”

### SUMMARY OF THE INVENTION

The present invention provides a method for treating glaucoma and other ophthalmic diseases caused by premature stop mutations. According to the present invention, aminoglycoside antibiotics are locally and/or systemically administered to a patient suffering from such a type of ophthalmic disease in order to overcome the stop mutation and allow sufficient amounts of functional protein to be expressed.

Without being bound to any theory, it is believed that aminoglycoside antibiotics work in bacteria by blocking protein synthesis at the level of tRNA charging on the ribosome. In eukaryotic cells, these antibiotics can lead to incorporation of an amino acid at a nonsense (stop) mutation and prevent premature protein translation termination.

### DETAILED DESCRIPTION OF THE INVENTION

According to the present invention, aminoglycosides are used to treat patients with genetically defined ophthalmic diseases that are caused by stop mutations within the coding region of a gene. Patients harboring these specific mutations can be identified by genotype testing using one of many different methods well know by those skilled in the art. For example, individuals with the Glu 368 Stop mutation in the GLC1A (MYOC) glaucoma gene can be identified by: obtaining a DNA sample from their blood or buccal (cheek) cells, PCR amplification of the representative region of the GLC1A gene using specific PCR primers, and SSCP (single-strand conformational polymorphism) analysis of the PCR amplicon; Stone, et al., “Identification of a Gene That Causes Primary Open Angle Glaucoma,” Science Vol. 275(5300) pp. 621 (1997); Fingert et al., “Analysis of Myocilin Mutations in 1703 Glaucoma Patients from Five Different Populations,” Hum Mol Genet, Vol. 8(5); pp. 899-905 (1999). A number of other methods such as DGGE (denaturing gradient gel eletrophoreis), ASO (allele specific oligonucleotide) hybridization, RFLP (restriction fragment length polymorphism), heteroduplex analysis, CCM (chemical cleavage of mismatches), PTT (protein truncation test), and RNase cleavage can also be used.

After a patient has been identified using the diagnostic methods described above, an aminoglycoside antibiotic compound is administered. Any ophthalmically acceptable aminoglycoside antibiotic compound can be used in the method of the present invention. Many ophthamically acceptable aminoglycoside antibiotic compounds are known. Such compounds include, but are not limited to, gentamicin; tobramycin; metilmicin; amikacin; kanamycins A and B; streptomycin; netlimicin; and neomycin.

The aminoglycoside antibiotic may be administered in a variety of ways, including systemically. Local administration is preferred, however. For example, the antibiotic could be administered topically in the form of solutions, gels or ointments. The antibiotic could also be administered intraocularly, incorporated in a drug delivery implant or combined with a sustained release vehicle, for example. Additionally, the antibiotic could be administered via periocular or subconjunctival injections. For purposes of the present application, “an ophthalmically acceptable composition comprising an aminoglycoside antibiotic compound” includes, but is not limited to, the drug delivery implant, injectable compositions, and solutions, gels or ointments referenced above. “Local administration” includes, but is not limited to, the topical, intraocular (e.g., intravitreal or subconjunctival) and periocular administration referenced above.

It is anticipated that the antibiotic therapy of the present invention would need to be administered over extended periods of time, in some cases even for the life of the patient. Though the antibiotic concentration would vary depending on the route of administration, compositions intended for topical administration according to the present invention generally will comprise 1% (w/w) or less, preferably 0.2-0.4% (w/w) of an aminoglycoside antibiotic compound.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A method for treating a patient diagnosed with an ophthalmic disease caused by premature stop mutations in a gene, wherein the method comprises administering to such patient a composition comprising an aminoglycoside antibiotic compound.

2. The method of claim 1 wherein the ophthalmic disease is glaucoma.

3. The method of claim 2 wherein the patient is diagnosed with a stop mutation in a gene selected from the group consisting of GLC1A and CYP1B1.

4. A method for treating a patient diagnosed with an ophthalmic disease caused by premature stop mutations in a gene, wherein the method comprises locally administering to the eye of such patient a composition comprising an aminoglycoside antibiotic compound.

5. The method of claim 4 wherein the ophthalmic disease is glaucoma.

6. The method of claim 5 wherein the patient is diagnosed with a stop mutation in a gene selected from the group consisting of GLC1A and CYP1B1.

7. The method of claim 5 wherein the aminoglycoside antibiotic is selected from the group consisting of gentamicin; tobramycin; metilmicin; amilcacin; kanamycins A and B; streptomycin; netlimicin; and neomycin.

8. The method of claim 5 wherein the composition is topically administered as an eye drop.

9. The method of claim 8 wherein the aminoglycoside antibiotic compound is present in the composition in an amount of 1% (w/w) or less.

10. The method of claim 9 wherein the aminoglycoside antibiotic compound is present in the composition in an amount of 0.2-0.4% (w/w).
